(19) United States
(12) Patent Application Publication
(10) Pub. No.: US 2013/0012446 A1
(43) Pub. Date: Jan. 10, 2013

(54) COMPOSITIONS AND MANUFACTURE OF MAMMALIAN STEM CELL-BASED COSMETICS

(75) Inventors: M. Rocio Sierra-Honigmann, Thousand Oaks, CA (US); Jaime Flores-Riveros, Thousand Oaks, CA (US); Martin Sierra, Newbury Park, CA (US)

(73) Assignee: AMERSTEM, INC, Newbury Park, CA (US)

(21) Appl. No.: 13/635,344

(22) PCT Filed: Mar. 18, 2011
(86) PCT No.: PCT/US11/29107

§ 371 Date: Sep. 14, 2012

Related U.S. Application Data
(60) Provisional application No. 61/315,623, filed on Mar. 19, 2010.

(51) Int. Cl. A61K 8/64; A61Q 19/08; A61K 8/65; C12P 21/00

(52) U.S. Cl. 514/17.2; 435/70.3; 514/21.2; 435/68.1; 514/20.9

(57) ABSTRACT

Methods of manufacturing collagen and other extracellular matrix components from SCCs are disclosed. The extracellular matrix components are useful in cosmetic applications, and can be manufactured free of immunogenic concerns and contaminants while controlling for other factors that commonly impact product quality and usefulness.

### FIELD OF THE INVENTION

This invention relates to the use of extracellular matrix components derived from stem cells in skin-related applications.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Skin care products for anti-aging and anti-wrinkle applications operate through a variety of mechanisms. This includes products that promote moisture retention for hydration of the skin surface, application of nutrients to nourish skin cells, and reducing exposure to noxious agents, among many others (for example, PCT App. No. PCT/US2010/044162 and U.S. Pat. No. 7,887,858). Skin tissue possesses inherent properties of self-renewal and regeneration as a result of complex biochemical interactions within different skin layer compartments (epidermis and dermis). The present invention relates to skin cosmetics and methods of manufacture, including cosmetics that feature constitutive extracellular matrix (ECM) components derived from cultured stem cells. Purified and isolated ECM from cultured stem cells also provides a consistent and renewable source of biologically active molecules, which can be used for anti-aging and anti-wrinkle applications by enhancing the regenerative capacity of the skin. Furthermore, manufacturing from cell cultures minimizes the potentially deleterious effects and problems posed by contamination, impurities and immunogenicity.

Constitutive ECM components from animal tissue, such as different kinds of protein collagens and elastin, glycoprotein fibronectin and laminin are widely used in the cosmetic, biomedical and pharmaceutical industries. Although these ECM proteins are typically extracted from pooled tissues, mainly from bovine origin, some human ECM products have been obtained from adult cadaveric tissues. These sources of ECM components pose risks to users from the presence of possible infectious and immunogenic agents. Further, animal or human sources of ECM components may vary in efficacy and consistency, due to variability in extrinsic environmental exposure (e.g. UV exposure, dietary intake) or intrinsic heterogeneity in individual or groups of source organisms. Such sources may suffer from molecular cross-linking due to UV exposure, or be degraded or excessively modified by non-enzymatic glycation.

Other ECM components, including for example, collagen and collagen-derived products also can be produced recombinantly (for example, U.S. Pat. No. 6,992,172). However, recombinant proteins can have different patterns of cross-linking and other post-translation modifications that are provided in living cells. Thus, recombinantly produced ECM components may lack the intricate molecular arrangements necessary for fully activating the regenerative capacity of the skin and halting the progressive degradation of skin compartments due to aging or environmental exposure.

A new source of ECM products is desirable in which active ECM components such as proteins, glycoproteins, and proteoglycans are produced in a controlled environment. This approach minimizes exposure to infectious and immunogenic agents, reduces environmental and biological variability, and improves efficacy with biologically compatible and biologically activated molecules. Aspects of the invention described herein provide methods of manufacture of ECM products from clonally expanded embryonic stem (ES) cells, adult stem cells or any other type of pluripotent or multipotent cell capable of self-renewal.

Embryonic stem cells are pluripotent cells that give rise to multipotent stem cells, which in turn are capable of differentiating into virtually all cell types in an organism. Embryonic stem cells possess properties of self-renewal that allow virtually unlimited propagation in cell cultures without differentiation. Embryonic stem cells are available from a variety of organisms including mice, primates (U.S. patent application Ser. No. 11/033,335; U.S. Pat. Nos. 5,843,780, 6,200,806, and 7,582,479), and humans (U.S. patent application Ser. No. 09/975,011; Thomson et al. “Embryonic Stem Cell Lines Derived from Human Blastocysts,” Science. 282 (1998): 1145-47). The differentiation and self-renewal properties of the ES cell provide a consistent and renewable source of biological material, which can be adapted for delivering biologically active molecules for use in anti-aging and anti-wrinkle applications.

Cultured ES cells can form embryoid bodies (EBs), which are small clusters of multipotent progenitor cells, some of which are already committed to a specific lineage. Therefore, EBs can be defined as organized stem cell-derived cell clusters containing progenitor cells partially committed to the various lineages originating from the 3 germ layers (endoderm, mesoderm, ectoderm). These clusters may contain both pluripotent and multipotent stem cells, herein referred to as a stem cell clusters (SCCs). Differentiation of ES cells into SCCs promotes expression, production and development of various ECM components, including proteins, glycoproteins, and proteoglycans, that are involved in skin maintenance and repair mechanisms. SCCs form spontaneously in cell cultures, following withdrawal of factors supporting pluripotency (e.g., growth factors, serum, matrix or adherence substrate) and in physical conditions supporting aggregation into clusters (e.g., semi-solid solutions, low adherence tissue culture surfaces, hanging drop suspension).

While ES cells and SCCs have been well-studied, the use of ECM components prepared or derived from cultured ES cells for cosmetic applications has not been recognized and developed. There remains a need in the art for consistent and renewable sources of ECM components from cultured ES cells. There also remains a need in the art for additional cosmetic formulations that achieve anti-wrinkle, anti-aging and other therapeutic and/or cosmetic benefits.

### SUMMARY

The following embodiments and aspects thereof are described and illustrated in conjunction with compositions and methods which are meant to be exemplary and illustrative, not limiting in scope. The present invention provides a method for obtaining at least one extracellular matrix component, the method comprising the steps of culturing mammalian embryonic stem cells (“ESCs”) to form a culture of ESCs, extracting from the culture of ESCs or differentiated ESCs the at least one extracellular matrix component. In one embodiment, the mammalian ESCs are murine ESCs. In another embodiment, the mammalian ESCs are cultured on feeder cells. In another embodiment, method further comprises the step of inducing differentiation in the culture of ESCs prior to the step of extraction. In another embodiment, method further comprises treating the culture of ESCs with dispase or collagenase prior to the step of inducing. In another embodiment, the inducing step further comprises transferring the culture of ESCs to a container under conditions which reduce the likelihood of adherence of the culture of ESCs to a surface of the container. In another embodiment, the inducing step further comprises inducing the culture of ESCs in a media solution. In another embodiment, method further comprises the step of rocking a container containing the culture of ESCs. In another embodiment, method further comprises inducing the culture of ESCs in a hanging drop. In another embodiment, the step of extracting further comprises contacting the culture of ESCs with a salt, a detergent and/or an acid, and separating the culture of ESCs from the at least one extracellular matrix component. In another embodiment, method further comprises the step of purifying the at least one extracted extracellular matrix component. In another embodiment, the step of purifying further comprises centrifugation, chromatography, precipitation, filtration and/or organic solvent extraction. In another embodiment, method further comprises the step of lyophilizing the at least one extracted extracellular matrix component. In another embodiment, the at least one extracellular matrix component is a collagen. In another embodiment, the method further comprises contacting the at least one extracellular matrix component with a protease. In another embodiment, the at least one extracellular matrix component is a proteoglycan. In another embodiment, the at least one extracellular matrix component is elastin. In another embodiment, the at least one extracellular matrix component is laminin or fibronectin.

Another embodiment of the present invention provides a composition comprising at least one extracellular matrix component extracted from a culture of mammalian embryonic stem cells (“ESCs”) or differentiated ESCs and a cosmetically-acceptable carrier. In one embodiment, mammalian ESCs are murine ESCs. In another embodiment, the mammalian ESCs are cultured on feeder cells. In another embodiment, the at least one extracellular matrix component is produced by a process, comprising culturing ESCs to form a culture of ESCs, inducing the ESCs to differentiate, and extracting from the culture of ESCs the at least one extracellular matrix component. In another embodiment, the composition further comprises treating the culture of ESCs with dispase or collagenase prior to the step of inducing. In another embodiment, the inducing step further comprises transferring the culture of ESCs to a container under conditions which reduce the likelihood of adherence of the culture of ESCs to a surface of the container. In another embodiment, the inducing step further comprises inducing the culture of ESCs in a media solution. In another embodiment, the step of extracting further comprises contacting the culture of ESCs with a salt, a detergent and/or an acid, and separating the culture of ESCs from the at least one extracellular matrix component. In another embodiment, the present invention further comprises the step of purifying the at least one extracted extracellular matrix component. In another embodiment purification step further comprises centrifugation, chromatography, precipitation, filtration and/or organic solvent extraction. In another embodiment, the composition further comprising the step of lyophilizing the at least one extracted extracellular matrix component. In another embodiment, the at least one extracellular matrix component is a collagen. In another embodiment, the at least one extracellular matrix component is treated with a protease. In another embodiment, the at least one extracellular matrix component is a proteoglycan. In another embodiment, the at least one extracellular matrix component is elastin. In another embodiment, the at least one extracellular matrix component is laminin or fibronectin.

The present invention further provides a method of manufacturing a composition comprising the steps of providing at least one extracellular matrix component extracted from a culture of mammalian embryonic stem cells (“ESCs”) or differentiated ESCs and adding a cosmetically-acceptable carrier to the at least one extracellular matrix component. In one embodiment, the mammalian ESCs are murine ESCs. In another embodiment, the at least one extracellular matrix component is a collagen. In another embodiment, the at least one extracellular matrix component is a proteoglycan. In another embodiment, the at least one extracellular matrix component is elastin. In another embodiment, the at least one extracellular matrix component is laminin or fibronectin.

The present invention also provides a method of treatment comprising the steps of providing a composition comprising at least one extracellular matrix component extracted from a culture of mammalian embryonic stem cells (“ESCs”) or differentiated ESCs, applying the composition to a subject, whereby the subject is treated. In another embodiment, the mammalian ESCs are murine ESCs. In another embodiment, the at least one extracellular matrix component is a collagen. In another embodiment, the at least one extracellular matrix component is a proteoglycan. In another embodiment, the at least one extracellular matrix component is elastin.

In another embodiment, the at least one extracellular matrix component is laminin or fibronectin.

The present invention further provides the use of a composition comprising at least one extracellular matrix component extracted from a culture of mammalian embryonic stem cells (“ESCs”) or differentiated ESCs in the manufacture of a cosmetic composition to treat a subject treatable by application of the composition.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 is a depiction of a process embodiment for the formation of SCCs. Cells from a stock of actively growing ES cells are transferred into culture media and dispersed into individual drops of media, which are then placed inverted in a culture plate. As cells within each drop grow, they form spherical multicellular aggregates herein referred to as Stem Cell Clusters or SCCs (also known as embryoid bodies). The SCCs are transferred to an adherent culture plate and further expanded in culture for 12 days. On day 15, the resulting SCCs are harvested and the ECM extracted.

FIG. 2 is a depiction of the presence of ECM elaborated by stem cells grown in culture under conditions favoring formation of SCCs. Murine SCCs were grown in culture for the indicated periods of time and then harvested for histological examination. Frozen sections were prepared and then processed for standard H&E staining, followed by visualization at two different magnifications (lower magnification: 4 days FIG. 2A, 10 days FIG. 2B, 15 days FIG. 2C; higher magnification: 10 days FIG. 2D, 14 days FIG. 2E). As the SCCs expand in culture, they produce extracellular matrix visible as an amorphous hyaline material interspersed in the intercellular space (arrows). Thus, SCCs represent a suitable source of ECM components elaborated by pluripotent and multipotent stem cells in culture.

FIG. 3 is a depiction of the major types of collagen produced by stem cells grown in culture in the form of SCCs, including collagen IV. SCCs were harvested after 14 days in culture as described in FIG. 2 and frozen sections were prepared for immunostaining using antibodies against collagen I (FIG. 3A), III (FIG. 3B) and IV (FIGS. 3C&D). Immune complexes were detected with Alexa Fluor® 594-conjugated goat anti-mouse IgG and visualized under a fluorescence microscope. It is evident that SCCs can produce the major types of collagens, including collagen IV. Collagen IV is a distinctive type of non-fibrillar collagen that forms sheet-like aggregates predominantly found in basement membranes and at the dermal-epidermal junction (DEJ). The DEJ is a specialized structure separating the epidermis and dermis, which plays a key role in the normal barrier function of the skin.

FIG. 4 is a depiction of an embodiment of the process for the extraction and fractionation of ECM components and incorporation of purified component fractions into formulations for cosmetic applications. Pluripotent ES cells are grown under culture conditions favoring formation of SCCs, which are then harvested and processed for extraction of collagen-enriched ECM components. Extracts are prepared either by solubilization with an organic acid (e.g. lactic, acetic) aided by gentle digestion with pepsin or by cell lysis using a combination of a non-ionic detergent and NH₄OH. The resulting collagen-enriched fractions are characterized by measuring the abundance of soluble collagen and/or hydroxyproline content. These purified extracts are then incorporated into suitable cosmetic formulations containing appropriate emollients and preservative agents and microencapsulated into liposomes or nanoparticle carriers. Cosmetic performance is then established in human volunteers by determining the safety and stability profile, as well as moisturization and wrinkle reduction efficacy.

FIG. 5 is a depiction of a biochemical analysis of ECM extracts prepared from SCCs confirming the presence of collagen IV and collagen I, components that represent abundant elements of the normal ECM. SCCs were harvested after 14 days in culture (see FIG. 2) and crude fractions prepared using a partial pepsin/acid extraction as described in FIG. 4. FIG. 5A, the protein composition of the extracts was initially assessed by electrophoretic fractionation of proteins (SDS-PAGE) in serially titrated samples, showing a predominant band with an apparent molecular mass of 50 kDa. FIG. 5B, the predicted peptides resulting from a pepsin digest of collagen IV indicate the presence of two 50 kDa peptides (P1 and P2), which originate from each of the α Col IV tropocollagen chains (α1 IV and α2 IV). Aumailley and Timpl, “Attachment of Cells to Basement Membrane Collagen Type IV,” J. Cell Biol. 103 (1986): 1569-1575. Peptide P3 is further digested into much smaller fragments that would migrate towards the bottom of the gel. FIG. 5C, the presence of peptides with collagen IV and collagen I immunoreactivity was determined in extract samples (AMS) by ELISA using specific anti-Col IV and anti-Col I antibodies. Signal detected in the AMS samples was compared to the serially-diluted standards to estimate the relative concentration of collagen IV- and collagen I-related peptides. These results suggest that soluble ECM extracts prepared from cultured SCCs contain collagens I and IV, providing independent biochemical evidence consistent with the immunological detection of these collagens in intact SCCs.

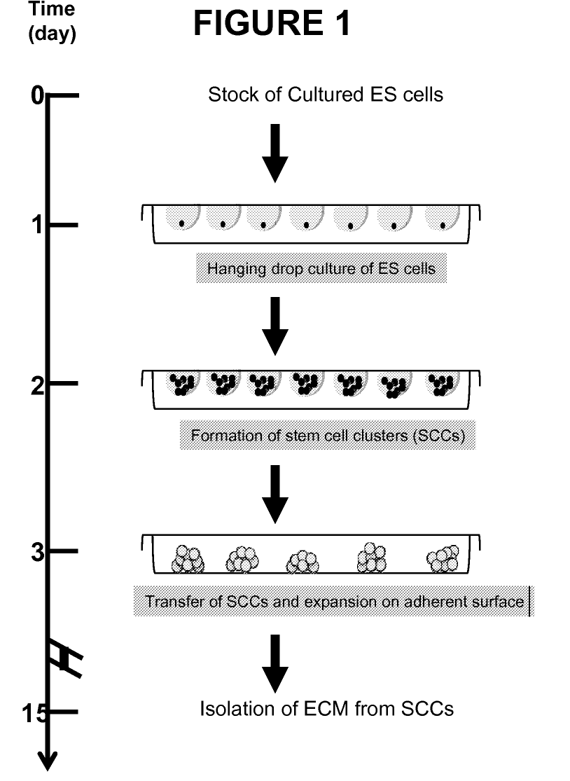

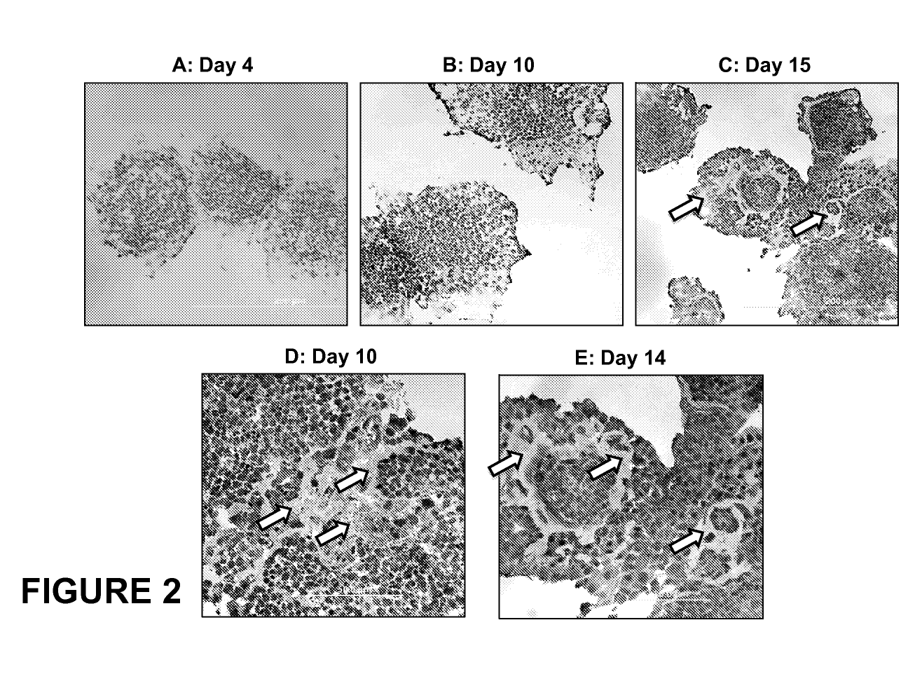

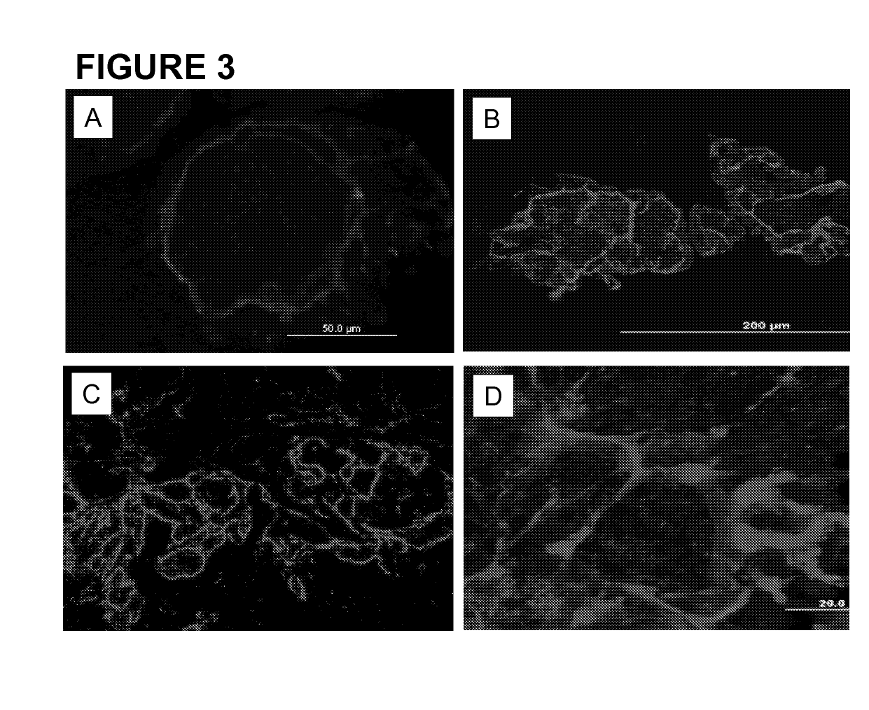

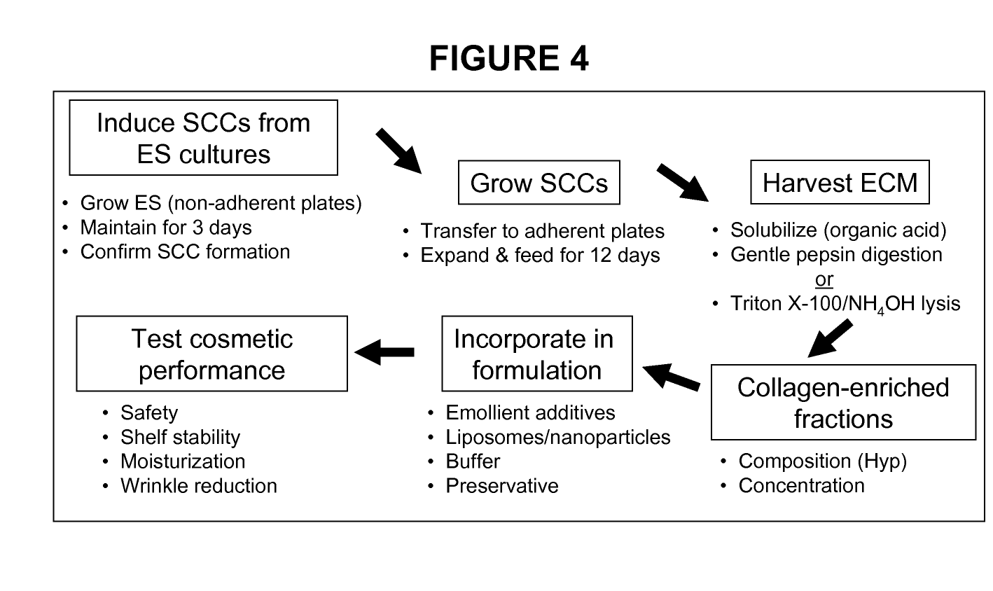

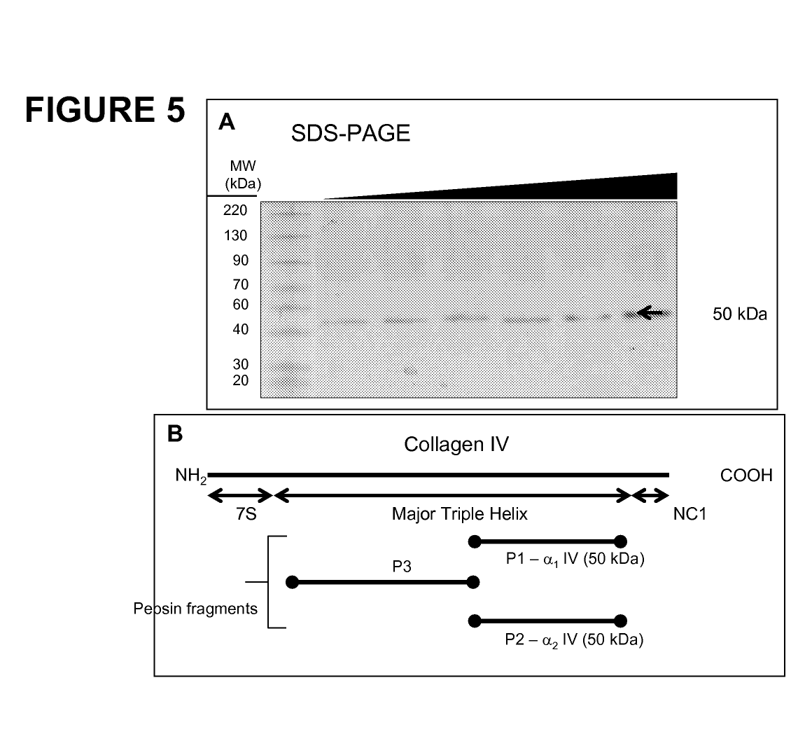

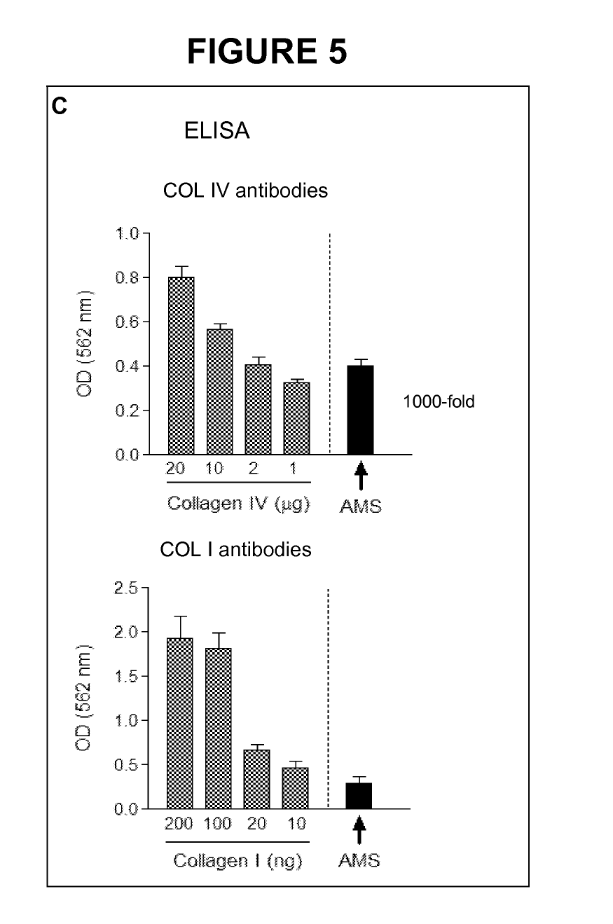

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The term “stem cells” as used herein, are cells that are not terminally differentiated and are therefore able to produce cells of other types. Stem cells are divided into three types, including totipotent, pluripotent, and multipotent. “Totipotent stem cells” can grow and differentiate into any cell in the body, including extraembryonic tissues (e.g. placenta) and thus, can form the cells and tissues of an entire organism. “Pluripotent stem cells” are capable of self-renewal and differentiation into any cell or tissue type, except extraembryonic tissues. In contrast to pluripotent cells, “multipotent stem cells” are unspecialized cells that can propagate indefinitely and differentiate into specialized cells with specific functions. In this respect, multipotent stem cells are essentially committed to differentiate into specific cell types. The term “stem cells”, as used herein when referring to cells obtained from any animal source, refers to either multipotent or pluripotent stem cells capable of self-renewal and differentiation. Examples include embryonic stem cells, induced pluripotent stem cells, induced multipotent stem cells, skin stem cells, umbilical cord, hematopoietic stem cells, neural stem cells, and mesenchymal stem cells. “Stem cells”, as used herein where referring to cells obtained from any non-animal source, refers to totipotent, multipotent, or pluripotent stem cells capable of self-renewal and differentiation. Examples include dedifferentiated cells obtained from plants, fruits and vegetables.

The term “embryonic stem cells” (ES cells) is used herein as it is used in the art and is a type of stem cell. Sources of ES cells include those derived from the inner cell mass of human blastocysts or morulae, which can be serially passaged as cell lines, and wherein use of the cell line for various methods and compositions does not directly involve the destruction of an embryo. Further exemplary stem cells include induced pluripotent stem cells (iPSCs) generated by reprogramming a somatic cell by expressing a combination of factors, including Oct 3/4, Sox2, c-Myc, Klf4, Nanog and lin28. The iPSCs can be generated using fetal, postnatal, newborn, juvenile, or adult somatic cells. As an alternative, potential induction of somatic cells into multipotent stem cells would further provide a suitable source of ECM materials.

Stem cells can be from any species of organism. Embryonic stem cells have been successfully derived in, for example, mice, multiple species of non-human primates, and humans, and embryonic stem-like cells have been generated from numerous additional species. Thus, one of skill in the art can generate embryonic stem cells from any species, including but not limited to, human, non-human primates, rodents (mice, rats), ungulates (cows, sheep, etc), among others. Similarly, iPSCs can be from any species. These iPSCs have been successfully generated using mouse and human cells. Furthermore, iPSCs have been successfully generated using embryonic, fetal, newborn, and adult tissue. Accordingly, one can readily generate iPSCs using a donor cell from any species.

Stem cells can be obtained from plant, fruit, and vegetables species, following the dedifferentiation of adult cells obtained from the plant, fruit, and vegetables species in cell cultures. When placed on a solid medium surface, such as agar, adult cells from the plant, fruit, and vegetables species are induced to dedifferentiate into pluripotent stem cells capable of self-renewal and differentiation into virtually every cell type found in the source plant, fruit, or vegetable.

The term “differentiation” of stem cells in general as used in the present invention means the change of pluripotent stem cells into multipotent cells committed to a specific lineage and/or cells having characteristic functions, namely mature somatic cells.

“Treatment” or “treating” refers to therapy, prevention or prophylaxis and particularly refers to the administration of medicine or cosmetics or the performance of medical or cosmetic procedures with respect to a subject. Treatment may be for prophylactic purposes to reduce the extent or likelihood of occurrence of a disease state, disorder or condition. Treatment may also be for the purpose of reducing or eliminating symptoms of an existing disease state, disorder, condition, or undesirable appearance. Treatment may directly eliminate infectious agents or other noxious elements causing a disease state, disorder or a condition. Treatment may alternatively occur through enhancement and stimulation of an organism's natural immune system, such as promoting or facilitating repair and regeneration of damaged or disease cells and/or tissue. Treatment may also occur by supplementing or enhancing the body's normal function, such as the formation of collagen.

“Subject” or “patient” refers to a mammal, preferably a human, in need of treatment for a condition, disorder or disease.

“Cosmetically effective amount” as used herein is the quantity of a composition provided for administration and at a particular dosing regimen that is sufficient to achieve a desired appearance, feel, and/or protective effect. For example, an amount that results in the prevention of or a decrease in the appearance and/or symptoms associated with an undesirable condition, such as wrinkles, fine lines, skin thinness, loss of skin elasticity or suppleness, or other characteristics of skin associated with aging, UV, chemical exposure, adverse climate (e.g., temperature, humidity), dietary intake, biological agents, environmental oxidants, among others.

The present invention relates to a method for isolating and purifying extracellular matrix components (ECM), through culturing of mammalian ES cells (native or induced), inducing the mammalian ES cell to form SCCs, and extracting from these SCCs at least one ECM component. The mammalian ES cell can be derived from any suitable mammal, including a primate, rodent, or a human ES cell. One stem cell embodiment includes a non-primate, mammalian ES cell. According to various methods, ES cells can be cultured on a layer of support feeder cells, but preferably, are cultured in the absence of feeder cells. The cells can be treated with enzymes during culture, including dispase or collagenase.

The ES cells are induced to form SCCs by any suitable technique. For example, the cultured cells can be transferred to a container under conditions which prevent adherence of the cells to a surface of the container. The cells can be rocked in a suitable media solution, or can be cultured in hanging drops to prevent adhesion to the cell culture surfaces. Alternatively, they can also be grown in a culture vessel made with a material that does not support adhesion of cultured cells.

The ECM components can be extracted from SCCs by a variety of methods, including treatment of the cell with a salt, a detergent or an acid, and then separating the cells from the ECM components. The extracted ECM components can then be further purified by any suitable method. For example, the extracted ECM components can be purified, enriched or concentrated by centrifugation, chromatography, precipitation, filtration or organic solvent extraction, or any combination of these and other biochemical techniques. Alternatively, ECM components can be extracted from cultured ES cells that are kept in their native, undifferentiated multipotent stage or cultured ES cells that are subjected to induced or spontaneous differentiation without necessarily being derived from SCCs. It is appreciated that crude preparations of multiple ECM components may be prepared through whole cell extracts obtained directly from cultured ES cells, partially differentiated ES cells not requiring SCC formation, or through cells obtained from SCC differentiation. However, purification of specific ECM components, substantially free of non-ECM molecules (e.g., nucleic acid, intracellular proteins), enhances efficacy of various compositions, by eliminating molecules possessing inert or interfering properties at the skin surface and increases safety by removing potentially immunogenic factors.

According to the methods, the ECM component can be a collagen-containing extract, wherein the extracted ECM is treated with a protease to purify the collagen. Suitable proteases include: papain, chymo-papain, bromelain, protease VIII, or protease X. The ECM component to be purified also can be a proteoglycan, including, for example, hyaluronic acid, chondroitin sulfate, or heparan sulfate. The ECM component also can be elastin, laminin or fibronectin, as well as any other previously functionally active elements that form part of the ECM produced by ES cells.

Aspects of the present invention also include compositions of at least one ECM component purified from an embryoid body according to the methods of the present invention. Such compositions are suitable for a variety of applications, including cosmetic applications. The components also can be used as matrix components or stimulants or inhibitors for cell culture.

Other aspects of the present invention also provide methods delivering to a subject a cosmetically effective amount of a composition of the present invention. The extracted or purified ECM components can also be used directly in a subject to neutralize or inhibit endogenous proteases (e.g. matrix metalloproteinases or MMPs), induce cell growth, enhance production of regenerative factors, or to create a niche for cell homing at desired tissues or organs. The ECM components also can be applied to treat skin disorders including scars, burn, abrasion, incision, contusion or laceration. The ECM components also can be applied to treat skin defects or deformations including folds, wrinkles, distensions, asymmetries and other defects that are correctible using ECM. For such treatments, the composition is typically delivered intradermally, subcutaneously, surgically or topically.

Embryonic Stem Cells.

Embryonic stem cells are unique cells capable of self-renewal and differentiation into cell types derived from all three embryonic germ layers (mesoderm, endoderm, and ectoderm). Embryonic stem cells are derived from the inner cell mass of mammalian blastomeres and can be grown as cell lines plated on either mitotically-inactivated fibroblasts “feeder” support cells or under feeder-free conditions using a support matrix (e.g., gelatin, matrigel, collagen). More recently, ES cells can be grown in chemically defined conditions without the use of animal serum, thereby eliminating the risk of exposure to xenogenic pathogens. Clinical grade human ES cells lines have also been established, wherein initial isolation and subsequent culturing of ES cells has been performed entirely without the use of non-human animal products. Ellerström et al., “Derivation of a Xeno-Free Human Embryonic Stem Cell Line,” Stem Cells. 24 (2006): 2170-6.

Generally, ES cells possess cellular morphology of round shape, large nucleolus and scant cytoplasm. Embryonic stem cells from different species can be characterized by various sets of markers associated with pluripotency, as known in the art. For example, undifferentiated mouse ES cells possess a compact, round, multi-layer cluster morphology and express several cellular markers associated with pluripotency. This includes transcription factors Oct-4, Sox-2, and Nanog, surface antigen SSEA-1, and high levels of alkaline phosphatase (AP) expression. In contrast, pluripotent human ES cells possess a sharp-edged, flat, tightly-packed colony morphology, although similar markers can be used to characterize pluripotency in human ES cells. This includes expression of Oct-4, Sox-2, and Nanog with high levels of AP expression, and surface antigens SSEA-3, SSEA-4, TRA-1-60, TRA-1-81. A variety of established biochemistry, cell and molecular biology techniques can be used to detect the expression of these pluripotent markers, including flow cytometry, reverse transcription PCR (RT-PCR), quantitative real-time PCR (qRT-PCR), western blotting, enzymatic staining, among others. Other techniques can establish the pluripotent capacity of cell cultures, including teratoma formation in immunodeficient mice. Forming teratomas in mice requires injection of pluripotent ES cells into immunodeficient mice and observing formation of differentiated cell types from all three embryonic germ layers.

Mouse embryonic stem cells have been widely available as established cell lines for over 20 years. Examples of established mouse cell lines include ES-057BL/6, J1, R1, R1/E, ESF 158, RW.4, AB2.2, B6/BLU, CE-1, CE3, and CCE. Further examples include those listed in databases for distributors such as ATCC, Jackson Laboratory, Taconic, among others. Today, various human ES cell lines are established and readily available for distribution from commercial and non-commercial sources, thereby eliminating the need for directly manipulating embryos as source materials. Examples of established ES cell lines include H1, H7, H9, H13, H14, HES3-6, CHB1-12, HUES1-66, BG01V, among many others. This also includes iPSC cell lines, such as DMD-IPS1, DMD-IPS2, DS1-IPS4, HD-IPS1, HD-IPS4, IPS(FORESKIN)-1, IPS(FORESKIN)-2, IPS(FORESKIN)-3, IPS(FORESKIN)-4, IPS(IMR90)-1, IPS(IMR90)-2, IPS(IMR90)-3, IPS(IMR90)-4, among many others. Further examples of human ES and iPSC cell lines include those registered in the University of Massachusetts International Stem Cell Registry.

Culturing Embryonic Stem Cells.

The methods of the present invention can be used with any mammalian ES cell line including new stem cell lines derived from mammalian blastocysts or any induced stem cells of somatic origin. In one embodiment, the stem cells are human ES cells, primate stem cells, rodent stem cells, bovine stem cells, or porcine ES cells. Importantly, ES cells are capable of self-renewal and can be propagated indefinitely in cell cultures, thereby providing a consistent and renewable source of biological material.

The selection of ES cell will depend on the application. For example, human embryonic stem cells may be the most desirable source for use in extracting collagen for injection. Where the use is topical, the stem cell source may be derived from another species, including murine or mammalian embryonic stem cells.

The ES cells can be maintained in culture according to suitable methods known in the art. It is advantageous to prevent ES cells from differentiating until it is desirable to induce formation of SCCs or any other manipulation resulting in differentiation of stem cells. Differentiated cells possess reduced proliferative capacity and diminished capacity to mature into various cell types. A number of methods of culturing both mouse and human ES cells are known and described in the art (for example, U.S. patent application Ser. No. 11/027,395 and 10/507,884; U.S. Pat. Nos. 7,455,983, 7,297,539 and 7,439,064). Briefly, ES cells can be cultured on a substrate of mitotically-inactivated support feeder cells or cultured under defined conditions in the absence of feeder cells (Ludwig et al., “Derivation of Human Embryonic Stem Cells in Defined Conditions,” Nature Biotechnology. 24 (2006): 185-187; Ludwig et al., “Feeder-Independent Culture of Human Embryonic Stem Cells,” Nature Methods 3 (2006): 637-646). Other techniques and methods for culturing stem cells can be found in Turksen, ed., Embryonic Stem Cells: Methods and Protocols, Humana Press (Totowa, N.J. 2002); Notarianni and Evans, Embryonic Stem Cells: A Practical Approach, Oxford University Press (U.S.A. 2006); and Robertson, ed., Teratocarcinomas and Embryonic Stem Cells: A Practical Approach, Oxford IRL Press (Washington D.C. 1987).

A variety of growth factors are utilized for the expansion and maintenance of undifferentiated ES cells without the use of support feeders. For example, mouse ES cells can be readily expanded in the presence of leukemia inhibitory factor (LIF) supplemented with serum or under chemically-defined conditions. Human ES cells require more complex solutions, but also can be expanded under feeder-free and/or chemically-defined conditions. One example includes the use of knockout serum replacement (KO-SR), basic fibroblastic growth factor (bFGF) on matrigel, as described in Xu et al., “Feeder-Free Growth of Undifferentiated Human Embryonic Stem Cells,” Nature Biotech. 19 (2001): 971-4. Other combinations include the use of Wnt3a (or Wnt/β-catenin pathway agonists), TGFβ, Activin A, and Nodal, in combination with bFGF (Nieden, ed., Embryonic Stem Cell Therapy for Osteo-Degenerative Diseases: Methods and Protocols, Humana Press (Totowa, N.J. 2010)). Another example includes use of April, BAFF (B cell activating factor), Wnt3a, insulin, transferrin, albumin, cholesterol, in combination with bFGF (Ludwig et al., 2006; Lu et al., “Defined Culture Conditions of Human Embryonic Stem Cells,” Proc. Nat. Acad. Sci., 103 (2006): 5688-5693).

Large Scale Production of Embryonic Stem Cells.

Embryonic stem cells can be grown in large scale cultures according to suitable methods known in the art. Generally, expansion of pluripotent stem cells and later differentiation into multipotent stem cells or specialized cell types depends on physiochemical environment, nutrients and metabolites and the presence/absence of growth factors. Polak and Mantalaris, “Stem Cells Bioprocessing: An Important Milestone to Move Regenerative Medicine Research into the Clinical Arena,” Ped. Res. 63 (2008): 461-466.

A well-established technique to provide a suitable physiochemical environment for large scale expansion of ES cells is the use of cell culture flasks (e.g., T75, T150, T175 flask) or cell trays. Thomson, Trends in Biotech. 25 (2007): 224-230. Embryonic stem cells grown in cell culture flasks or trays can adhere directly to an untreated cell culture surface, can be deposited on a layer of support matrix (e.g., gelatin, matrigel, collagen) to provide a semi-adherent state of attachment, or are grown in suspension without attachment. Traditionally, advantages of “two-dimensional” culture systems include simplicity, ease of handling, and low cost. Several cell culture flasks or trays can be combined together to form cell “factories”, thereby providing an easy and straightforward means to grow progressively larger numbers of cells. Placzek et al., “Stem Cell Bioprocessing: Fundamentals and Principles,” J. R. Soc. Interface. 7 (2009): 209-232. A further advantage includes convenient access to cells for harvesting or addition/supplementation of nutrients and metabolites. Additionally, cell culture flasks or trays provide few physical interfaces/openings, which lowers the risk of contamination or infiltration of contaminating particles. Often, cell culture flasks or trays are grown in static cell cultures, wherein diffusion is the primary means for mass transport of nutrients, metabolites, oxygen and other factors. Static culturing imparts little or no shear mechanical stress on cells, thereby maintaining cell viability, morphology and integrity.

Static cell culture techniques may be modified or adapted to provide physical or biomechanical features to improve expansion of undifferentiated ES cells or to promote development of specific cellular phenotypes. Examples of physical features include use of natural or synthetic scaffolds, which increase available surface area for cellular attachment and growth. Use of “three-dimensional” culture surfaces thereby proves higher cell densities in the expansion of undifferentiated ES cells. Similar improvements can be obtained through modification of the cell culture vessel surface, including use of recessed and/or elevated patterns, grooves, micro- and nano-chambers to increase surface area for cellular attachment (Thomson, 2007).

Biomechanical features include dynamic culture conditions to improve delivery of nutrients, metabolites, oxygen and other factors involved in stem cell growth and maintenance. Whereas static culturing relies primarily on diffusion for mass transport, dynamic culture conditions, enhance mass transport by altering fluid velocity in a cell culture (Thomson, 2007). Common examples include perfusion and stirring of cell culture media.

Use of dynamic cultures via stirring, has been reported to lead up to a 10-fold increase in cell density compared to traditional methods (Zandstra et al., “Stem Cell Bioengineering,” Ann. Rev. Biomed. Eng. 3 (2001): 275-305). A limitation of dynamic culturing conditions is creation of shear stress (i.e., the force exerted over cells due to the flow of media), which may lead to deleterious effects on stem cell viability if media velocity is too high. In contrast, low media velocities have been reported to result in cell clumping, which lowers overall mass transport conditions. Other techniques known in the art rely on microcarriers or encapsulation of cells to capture various features of both static (i.e., cell attachment with reduced shear stress) and dynamic (i.e., higher mass transport) cell culturing techniques.

Stem Cell Clusters.

Cultured stem cells can be induced to form SCCs, which are partially differentiated clusters of ES cells that spontaneously form following removal of pluripotent support factors and under physical conditions promoting cell aggregation. Differentiation of ES cells into SCCs promotes the expression, production and development of ECM components involved in skin maintenance and repair mechanisms, including proteins, glycoproteins, and proteoglycans. Differentiating ES cells into SCCs results in a loss of expression of ES cell pluripotent markers and induced expression of gene markers associated with multipotent cells derived from all three embryonic germ layers (ectoderm, endoderm, and mesoderm). A viable alternative to induced differentiation without formation of SCCs entails the use of appropriate culture conditions that directly promote multipotency via germ layer differentiation in the original ES culture. These differentiated cells are also regarded as a suitable source of ECM materials.

One of skill in the art can select a method appropriate for the type of mammalian ES cell being used. For example, SCCs can be formed from murine ES cells according to the methods described in Doetschman et al., “The In Vitro Development of Blastocyst-Derived Embryonic Stem Cell Lines: Formation of Visceral Yolk Sac, Blood Islands and Myocardium,” J. Embry. Exper. Morph. 87 (1985): 27-45; Keller, “In Vitro Differentiation of Embryonic Stem Cells,” Curr. Op. Cell Biol. 7 (1995): 862-869; and U.S. Pat. No. 5,914,268. An SCC can be formed, for example, by culturing a murine ES cell in an SCC cell medium that includes platelet-poor fetal bovine serum, preferably from about 1 day to about 7 days. Alternatively, others commonly used methods involve removal of LIF and serum to eliminate factors supporting ES cell pluripotency, coupled with physical methods to promote cell aggregation (e.g., hanging drop suspension, low adherence tissue culture surface, semi-solid solutions such as methylcellulose).

When the ES cells are primate ES cells, SCCs can be formed by suitable methods known in the art. Similar to mouse ES cells, human ES cells also spontaneously form SCCs when factors supporting pluripotency are removed, in the absence of serum and/or with the use of media and culture vessels which limit adherence to tissue culture surfaces (for example, U.S. Pat. No. 6,602,711). Briefly, ES cells growing on a substrate, such as feeder cells, are removed from the substrate and cultured under conditions that prevent adherence to a new container and which favors formation of SCCs. Examples include use of petri dishes, low adherence tissue culture surfaces, semi-solid solutions such as methylcellulose, hanging drop suspension, among others (Iskovitz-Eldor et al., “Differentiation of human Embryonic Stem Cells into Embryoid Bodies Comprising the Three Embryonic Germ Layers,” Mol. Med. 6 (2000): 88-95; Yang et al., “Novel Method of Forming Human Embryoid Bodies in a Polystyrene Dish Surface,” Biomacromolecules, 8 (2007): 2746-2752.) Differentiated SCCs can be removed from the substrate by mechanical force (e.g., centrifugation, physical separation) with or without the use of dissociating enzymes.

Primate ES cells (e.g., Rhesus or human, U.S. Pat. No. 5,843,780; Thomson et al., “Embryonic Stem Cells Lines Derived From Human Blastocysts,” Science. 282 (1998) 1145-1147) are cultured on mitotically inactivated (3000 rads γ-radiation) mouse embryonic fibroblasts, prepared at 5×10⁴ cells/cm² on tissue culture plastic previously treated by overnight incubation with 0.1% gelatin (Robertson, 1987). Culture medium consists of 79% Dulbecco's modified Eagle medium (DMEM; 4500 mg of glucose per liter; without sodium pyruvate), 20% fetal bovine serum (FBS), 0.1 mM 2-mercaptoethanol, 1 mM L-glutamine and 1% nonessential amino acid stock (GIBCO).

One allows colonies to form clumps over a period of hours. ES cell colonies can then be removed from the tissue culture plate using physical or chemical methods that keep the ES cells in clumps. For dispase or collagenase removal of ES cell colonies from the culture plate, the culture medium is removed from the ES cells. Dispase (10 mg/ml in ES culture medium) or collagenase (1 mg/ml solution in DMEM or other basal medium) is then added to the culture plate. The culture plates are returned to the incubator for 10-15 minutes.

After dispase treatment the colonies can either be washed off the culture dishes or will become free of the tissue culture plate with gentle agitation. After collagenase treatment the cells can be scraped off the culture dish with a 5 ml glass pipette. Some dissociation of the colonies occurs, but this is not sufficient to individualize the cells. After chemical removal of the cells from the tissue culture plate, the cell suspension is centrifuged gently for 5 minutes, the supernatant is removed and discarded, the cells are rinsed, and the cells are resuspended in culture medium with or without serum.

Mechanical removal of the cells is achieved by using a pulled glass pipette to scrape the cells from the culture plate. Cell clumps can be immediately resuspended, without centrifugation, in fresh tissue culture medium.

Once colonies are removed from the tissue culture plate, the ES cells should remain in suspension to promote SCC formation. This can be achieved by, for example, gently and continuously rocking the cell suspension. Cell suspensions are aliquoted into wells of 6-well tissue culture dishes, placed inside a sealed, humidified isolation chamber, gassed with 5% CO₂, 5% O₂ and 90% N₂ and placed on a rocker. The rocker is housed inside an incubator maintained at 37° C. The culture plates can be rocked continuously for at least 48 hours and up to 14 days.

Every 2 days, the plates are removed from the rocking device, the culture medium is removed, and fresh culture medium is added to the cells. The culture dishes are then returned to the rocking environment. Cells will also remain in suspension when cultured in suspension culture dishes without rocking, or when cultured in the absence of serum, which provides attachment factors. All cells are cultured at about 37° C., in a humidified, controlled gas atmosphere (either 5% CO₂, 5% O₂ and 90% N₂ or 5% CO₂ in air).

Following culture in suspension for up to 11 days, SCCs are dispensed by mechanical or chemical means and can be allowed to reattach to tissue culture plates treated with gelatin or matrix, in ES medium. Displaced, plated SCCs will form flattened monolayers and can be maintained by replacing medium every 2 days.

Extracellular Matrix Components.

While the examples provided describe ECM extraction from differentiated cells obtained via SCC formation, it is appreciated that such techniques are readily understood to be applicable to cultured ES cells or partially differentiated ES cells not requiring SCC formation. The ECM either produced by via SCCs formation or otherwise stated in accordance with alternate embodiments described herein, has a number of components, including structural proteins: collagen and elastin, glycoproteins: laminin and fibronectin; proteoglycans: hyaluronic acid, chondroitin sulfate, heparan sulfate; and other factors useful in the maintenance and regeneration of the skin. According to the methods of the present invention, the ECM derived from the SCCs can be used as a crude preparation, or can be further purified to individual components or fractions containing multiple components.

Extraction of the ECM can be accomplished by suitable techniques known to one of skill in the art. For example, methods for purifying ECM are described in Current Protocols in Cell Biology. John Wiley & Sons, 1998, sections 10.4, 10.9. Depending on the desired application, ECM preparations can be made in two and three-dimensional forms.

For example, a crude preparation of ECM can be prepared by treating the cultured cells with a dilute basic solution or a detergent. For example, the SCCs can be treated with 0.01 N NaOH or 0.1% triton-X. The cells can be removed from the solution by filtration. The resulting solution is highly enriched in the matrix components.

Alternatively, the SCCs can be homogenized in a salt solution (for example, in 3.6 M NaCl). The solute is centrifuged, and the insoluble material preserved after centrifugation at 10,000 rpm. The extraction with 3.6 M NaCl is repeated until no extractable material is observed by protein assays (Biorad analysis). The insoluble material is then extracted with DNAse (0.1%) and RNAse (0.1%), and finally 0.1% Triton X.

In other forms, crude preparations of ECM extracts may be prepared through whole cell extracts. In one example, whole cell extracts may be obtained by directly lysing cells without fractioning or removal of non-ECM components. Such whole cell extracts thereby contain not only ECM components, but other cellular structures and molecules, including nucleic acids, lipids, sugars, intracellular proteins, among others. However, it is further appreciated that purification of specific ECM components, wherein a purified composition is substantially free of non-ECM components may enhance efficacy by eliminating molecules possessing inert or interfering properties at the skin surface, while increasing safety by removing potentially immunogenic factors.

Purified preparations of ECM can be used to form a gel matrix for cell culture. Methods for the preparation of such a matrix are described in Current Protocols in Cell Biology. John Wiley & Sons, 1998, Unit 10.3.

Collagen Purification.

Collagen can be purified by any method known to one of skill in the art. For example, collagen can be purified by the methods described in Current Protocols in Cell Biology. John Wiley & Sons (New York, N.Y. 1998), Sec. 10.2.4. Briefly, homogenized cells from the embryoid body are homogenized repeatedly in 2 M guanidine followed by centrifugation. The supernatant is dialyzed to remove the guanidine.

Purified ECM or crude preparations of collagen can be further purified by enzymatic treatment with one or more proteases. For example, the ECM can be digested using papain, chymo-papain, bromelain, protease VIII, or protease X.

Either with or without an enzymatic treatment, the ECM can be further purified using any technique known to one of skill in the art. For example, the components can be separated by centrifugation, chromatography, precipitation, and other techniques for separating biological molecules.

Laminin-1 Purification.

Laminin-1 can be purified by suitable techniques known to one of skill in the art. For example, the methods described in Current Protocols in Cell Biology, Sec. 10.2.3 can be used. Briefly, the SCCs are homogenized in a 3.4 M NaCl solution. After centrifugation at 8000×g, the pellet is retained and suspended in 0.5 M NaCl. After centrifugation, the supernatant is retained and laminin-1 is purified by precipitation with ammonium sulfate, added to 30% saturation. The pellets containing laminin-1 are resuspended in a buffer solution and dialyzed to remove the ammonium sulfate. Laminin1 is then precipitated by bringing the NaCl concentration to 1.7 M, followed by centrifugation.

Stem Cells From Plants, Fruit, and Vegetables.

Stem cells have also been obtained from dedifferentiation of adult cells obtained from plants, fruit, and vegetables. Briefly, adult cells from these non-animal sources, can be placed in cell cultures on solid media surfaces composed of various ingredient promoting dedifferentiation. Induction into a callus, a mass of undifferentiated cells in cluster form, can occur in two to three weeks, and can continue to be cultivated until complete dedifferentiation of the adult cells is fully achieved (U.S. patent application Ser. No. 12/148,241). Calluses may be mechanically or chemically dissociated and grown in suspension media to provide greater numbers of cells for scale-up applications. As an example, dedifferentiated cells have been obtained from apples, such as Malus domestica. Extracts obtained from Malus domestica may be prepared for the purpose of cosmetic applications and have been shown to promote growth and proliferation of umbilical cord stem cells, hair follicle maintenance, and skin-related uses. Other examples of extracts from plants, fruits, and vegetables have been obtained from alpine rose, Rhododendron ferrugineum, grape, Vitis vinifera, and rasberry, Rubus idaeus.

Skin-Related Applications.

Skin consists of an outer layer of epidermis and an inner layer of dermis. The epidermis is made up of stratified squamous epithelium and is separated from the dermis by a specialized, underlying structure called basal lamina. The basal lamina is a layer of ECM on which the epithelium sits. The ECM of the basal lamina consists of several biomolecular components including collagens, proteoglycans and glycoproteins. Representative examples of collagens in the basal lamina include type IV collagen, examples of proteoglycans include hyaluronic acid, chondroitin sulfate, heparan sulfate, and entactin, while examples of glycoproteins include laminin and fibronectin. The heterogeneous molecules of the ECM provide structural integrity and biotrophic support for the maintenance and regeneration of surrounding tissues, further including the activity of skin stem cells within and below the basal lamina. As an example of the multi-faceted role resulting from interactions of various ECM components, various forms of protein collagens (e.g., Collagen I-VI) attach to negatively-charged proteoglycans (e.g., chondroitin sulfate, and heparan sulfate) and attract water molecules via osmosis to hydrate the ECM and surrounding cells.

This compartment also serves as a reservoir for growth factors and nutrients necessary for cell survival and maintenance. Anchoring to glycoproteins (e.g., fibronectin, laminin) tethers matrix components to cell surfaces, thereby providing signaling through associated receptors, including fibronectin-integrin and laminin-laminin receptor signaling. Signaling among ECM components serves to promote the continued production of ECM, while regulating expression and release of additional growth factors and nutrients from fibroblasts situated in the inner dermis and skin stem cells located within the basal lamina and epithelia. In addition to their presence in the basal layer of the epidermis, skin stem cells typically reside within niche structures associated with hair follicles. Fuchs et al., “Socializing With the Neighbors: Stem cells and Their Niche,” Cell. 116 (2004): 769-78; Fuchs, “The Tortoise and the Hair: Slow-Cycling Cells in the Stem Cell Race,” Cell. 137 (2009): 811-9. Since skin tissue is constantly regenerated during the life of an organism, these skin progenitor cells play a central role in the maintenance, repair and replacement of surrounding tissues. Gago, et al., “Age-Dependent Depletion of Human Skin-Derived Progenitor Cells,” Stem Cells. (27) 2009: 1164-72. However, these specialized skin progenitor cells also can suffer damage and depletion as a result of age and environmental insults.

Existing further within this context, type IV collagen is the predominant collagen present in the basal lamina. Khoshnoodi et al., “Mammalian Collagen IV”. Microsc. Res. Tech. 71 (2008): 357-70. Uniquely among collagens, type IV collagen is anchored through laminin, signals through laminin receptors, and due to the presence of additional C-terminus amino acids, lacks a glycine residue motif commonly found in other collagens. This causes formation of sheets of collagen IV characteristic of the basal lamina, in contrast to the triple-helical fibrillar structure characteristic of other forms of collagens. Berisio et al., “Crystal Structure of the Collagen Triple Helix Model [(Pro-Pro-Gly)(10)(3),” Protein Sci. 11 (2002): 262-70.

Skin aging is the result of cumulative alterations in skin structure, barrier function and appearance. These alterations are due to a combination of intrinsic chronological factors (e.g., advanced age) or extrinsic environmental exposure (e.g., UV, chemical exposure, temperature humidity, dietary intake, etc.). Wrinkles and thinning of the skin results from atrophy of the ECM components in the epidermis and dermis, including induction of ECM degrading enzymes such as matrix metalloproteinases (MMPs). Fisher et al., “Pathophysiology of Premature Skin Aging Induced by Ultraviolet Light,” New. Eng. J. Med. 337 (1997): 1419-28. Matrix metalloproteinases are a family of approximately two dozen proteases, which are specific for degrading particular extracellular components. Examples include collagenases (MMP-1, MMP-08, MMP-13, MMP-14, and MMP-18), which target triple-helical fibrillar collagens, and genlatinases (MMP-2 and MMP9), which are capable of degrading type IV collagen and gelatin. Prolonged induction and activation of MMPs leads to depletion and fragmentation of skin collagen, a reduction in collagen synthesis, depletion of growth factors and nutrients within reservoirs providing biotrophic support for skin cells, and diminished support from dermal fibroblasts and skin stem cells within the basal lamina and epithelia.

An additional mechanism leading to changed appearance of the skin is the combined effects of enzymatic and non-enzymatic cross-linking in relation to the turnover of ECM components such as collagen and elastin. Avery and Bailey, “Enzymic and Non-enzymic Cross-Linking Mechanisms in Relation to Turnover of Collagen: Relevance to Aging and Exercise,” Scand. J. Med. Sci. Sports 15 (2005): 231-40. Enzymatic cross-linking results from the catalytic activity of various enzymes, such as lysyl hydroxylase, lysyl oxidases, prolyl-hydroxylases, and are involved in hydroxylation of lysine residues in ECM components. In turn, catalytic activity leads to formation of di-valent and tri-valent cross-links, which bind long rod-like molecules within protein tissue fibers to reduce movement and slippage, thereby providing core mechanical strength to the fibers. Robins, et al., “The Chemistry of Collagen Cross-Links,” J. Biochem., 131 (1973): 771-80. Enzymatic cross-linking plays a vital role in the natural growth, maturation and turnover of skin proteins and establishment of its structural integrity. The other type of cross-linking, non-enzymatic cross-linking, is adventitious (i.e., occurring through external factors) and a prime example advanced aging effects, since the long half-life of proteins in the skin increases opportunities for such external factors (e.g. UV exposure, dietary intake) to produce deleterious effects associated with non-enzymatic cross-linking Baily et al., “Non-Enzymic Glycation of Fibrous Collagen Reaction Products of Glucose and Ribose,” J. Biochem. 305 (1995): 385-90. In contrast to enzymatic cross-linking, non-enzymatic cross-linking does not involves enzyme activity, but instead, results from glyco-oxidation (glycation) and lipo-oxidation reactions. Paul and Bailey, “Glycation of Collagen: The Basis of its Central Role in the Late Complications of Ageing and Diabetes,” Int. J. Biochem. 28 (1996): 1297-1310. A hallmark of the process is the formation of an intermediate Schiff base and Amadori rearrangement product, both of which undergo oxidation to form stable end-products known as advanced glycation end-products (AGE). (Avery and Bailey, 2005). Examples of AGE include FFI, pentosidine, NFC-1, malondialdehyde, among others. Importantly, the non-enzymatic cross-linking leads to intermolecular (e.g., interfibrillar) cross-linking between proteins and interferes with reactivity with other ECM components, thus reducing the optimal mechanical and effective functional properties of proteins within the skin. Thus, measurement of AGE products serves as a direct measure of the degree of glycation occurring in a sample of collagen and a proxy for the quality and integrity of ECM as a whole. The nature and extent of cross-linking in various skin proteins can be measured by a variety of techniques, including immunodetection of intermediate and end products of enzymatic and non-enzymatic processes, HPLC-based separation, optical detection of reactive species and presence/absence of associated by-products (e.g., CML and pyrraline), among others. Since ECM components obtained from SCCs are freshly made from cultured cells, there is a reduced degree of undesirable cross-linking present, particularly with respect to non-enzymatic glycation, thus possessing an important advantage over products obtained from animal sources.

Without being bound by any particular theory, the inventors believe that application of ECM purified/or obtained from ES cells will reverse or limit the deleterious effects of skin aging, through improved moisturization, neutralization of harmful enzyme degradation, and regeneration of skin components. First, application of ECM to the skin surface or within the epidermis, provides a source of negatively charged proteoglycans to increase retention of water for improved moisturization and hydration of the skin. Second, greater concentrations of ECM components, such as collagen, serve as enzyme substrates to neutralize or reduce the effects of MMP-degrading activity on cell and tissue surfaces. Third, enhancing levels of ECM components within the epidermis may activate signaling pathways associated with the normal regeneration and repair mechanism within skin tissues, including enhancing the response from fibroblasts and skin stem cells. Fourth, the ECM derived from cultured stem cells is freshly made, devoid of chemical cross-links or oxidative damage and is actively engaged in tissue development. Thus, this ECM may have inherent properties that would be desirable to delay skin aging and promote skin renewal.

Cosmetics.

Fractions of ECM can be processed and used in the form of solid powders, aqueous solutions (i.e., gels), partially emulsified aqueous solutions, or emulsifications (i.e., creams and lotions). Following isolation and purification, ECM components may be air-dried or freeze-dried in combination with heating/cooling, vacuum aspiration, centrifugation, and addition of salt or stabilizers to facilitate removal of moisture (for example, U.S. Pat. No. 7,115,388). Solid dried material may be ground or pummeled for longer-term storage or use in bulk industrial-scale manufacturing. Aqueous solutions can be formed from ECM, since the proteins and polypeptide chains in solution readily bind to each other via hydrogen bonding or through dispersion forces to form a three-dimensional mesh, wherein gel formation occurs. Addition of lipophilic components through mixing or stirring provides a partially emulsified aqueous solution, wherein a proportion of aqueous solution and oil component provides improved efficacy of skin cell growth, maintenance, and regeneration, with other desirable colligative properties such as improved adhesion and spreadability (i.e., extensivility). Various additives can be further provided in aqueous or oil components, including preservatives, pH adjusters, moisturizer, germicide, anti-inflammatory agent, dye, aromas, fragrances, antioxidants, ultraviolent absorbent, vitamin, alcohol, carbohydrates, or other components routinely used in skin care applications.

Extracellular components from SCCs can be formulated into a variety of cosmetic products. There are several benefits of incorporating stem cell extracts, including ECM components, as active ingredients in cosmetic products. Such extracts are typically colorless (or white), odorless, water-soluble, maintain stability and activity across a range of physiologically relevant pHs (i.e., 4-0-8.0), cosmetically effective as small amount of total product volume (i.e., 0.4%-1.0%), soluble and miscible. Collagen, for example, can be used for both topical, transdermal and internal applications.

Use of extracellular components derived from ES cells differentiated into SCCs has a number of advantages over current animal sources: 1) cultured ES cells can be maintained with reduced exposure to pathogens and infectious agents under laboratory conditions, eliminating reliance on animal or cadaveric sources possibly tainted with viruses, prions, or other disease causing agents; 2) extracellular membrane components derived from ES cells are a consistent and renewable source of biologically active ECM components, unaltered by the extrinsic factors such as environmental exposure or intrinsic biological variability, which affect animal or human sources of ECM. For example, ES cell-derived ECM has reduced cross-linkage, less oxidative damage, and lowered non-enzymatic glycation; 3) deriving ECM components from in vitro cultured ES cells provides critical post-translational modifications necessary for biological compatibility and activity, thereby improving efficacy for anti-aging and anti-wrinkle applications; and 4) ES cell-derived ECM is homogeneous and pathogen-free. This ECM may be prepared in vitro using a reproducible method of production and extraction.

### Various Embodiments

Further described herein is a method for obtaining at least one ECM component. In one embodiment, the method comprises the steps of culturing a quantity of mammalian ES cells, inducing the quantity of mammalian ES cells to form one or more SCCs and extracting from the one or more SCCs the at least one ECM component. In another embodiment, the ES cell line has been derived without exposure to non-human animal products. In one embodiment, the mammalian ES cells are murine ES cells. In another embodiment, the mammalian ES cells are human ES cells. In another embodiment, the mammalian ES cells are induced pluripotent stem cells obtained from adult somatic cells. In another embodiment, the mammalian ES cells are cultured on feeder cells. In another embodiment, the mammalian ES cells are cultured in serum-free conditions. In another embodiment, the mammalian ES cells are cultured in chemically defined conditions. In another embodiment, the quantity of mammalian ES cells are treated with dispase or collagenase prior to the step of inducing formation of SCCs. In another embodiment, the ES cells are grown in a plurality of tissue culture flasks or cell trays.

The present invention further provides methods to improve SCC formation. In one embodiment, the inducing step further comprises transferring the quantity of mammalian ES cells to a container under conditions that reduce the likelihood of adherence of the mammalian ES cells to a surface of the container. In another embodiment, the inducing step is performed in a media solution. In another embodiment, the media solution is a semi-solid solution. In another embodiment, the media solution is serum-free. In another embodiment, the media solution comprises platelet-poor serum. In another embodiment, there is an additional step of rocking the container to reduce the likelihood of adherence. In an alternative embodiment, the inducing step uses mammalian ES cells in a hanging drop. In various embodiments, the time period of induction to form SCCs is 1, 2, 3, 4, 5, 6, 7, 8, 9, 10, 11, 12, 13, or 14 day(s). In various embodiments, the SCCs are formed and are maintained as SCCs for a period of 1, 2, 3, 4, 5, or 6 day(s)s. In various embodiments, the SCCs are formed and are maintained as SCCs for a period of 1, 2, 3, 4, 5, 6, 7, 8, 9, 10 week(s).

The present invention further provides various methods to extract the extracellular component. In one embodiment, the step of extracting further comprises contacting the quantity of mammalian ES cells with a salt, a detergent and/or an acid, and separating the quantity of mammalian ES cells from the at least one ECM component. In another embodiment, the step further comprises the step of purifying the at least one extracted ECM component. In another embodiment, the step further comprises the step of purifying the at least one extracted ECM, comprising centrifugation, chromatography, precipitation, filtration and/or organic solvent extraction. In another embodiment, the step further comprises the step of lyophilizing the at least one extracted ECM component. In one embodiment, the at least one ECM component is a collagen. In another embodiment, the at least one ECM component is collagen IV. In another embodiment, the step further comprises contacting the at least one ECM component with a protease. In a different embodiment, the at least one ECM component is a proteoglycan. In one embodiment, the at least one ECM component is elastin. In another embodiment, the at least one ECM component is hyaluronic acid, chondroitin sulfate, or heparan sulfate. In a different embodiment, the at least one ECM component is a glycoprotein. In another embodiment, the at least one ECM component is laminin or fibronectin. In another embodiment, the at least one ECM component is substantially free of AGE. In various embodiments, the at least one ECM component comprises a quantity of AGE less than at least about 0.1%, at least about 0.5%, or at least about 1%, and may be as great as or more than about 5%, or about 10%, or about 15%, or about 20%, or about 25%, or about 35%, or about 50%, or about 80%, or about 90%. In various embodiments, the AGE is FFI, pentosidine, NFC-1, or malondialdehyde. In another embodiment, the ECM component is not substantially cross-linked.

The present invention further provides compositions derived from SCCs. In one embodiment, the composition comprises at least one ECM component extracted from SCCs and a cosmetically-acceptable carrier. In one embodiment, the at least one ECM component is a collagen. In a different embodiment, the at least one ECM component is produced by a process, comprising culturing a quantity of mammalian ES cells, inducing the quantity of mammalian ES cells to form one or more SCCs and extracting from the one or more SCCs the at least one ECM component. In one embodiment, the composition is derived from undifferentiated ES cells. In another embodiment, the composition is derived from partially differentiated ES cells not requiring SCC formation.

In various embodiments, the composition comprises one or more ECM components. In various embodiments, the composition comprises one or more ECM components selected from the group consisting of collagen, elastin, hyaluronic acid, chondroitin sulfate, heparan sulfate, laminin or fibronectin, and combinations thereof. In various embodiments, the composition comprises one or more ECM components of at least about 0.1%, at least about 0.5%, or at least about 1%, and may be as great as or more than about 5%, or about 10%, or about 15%, or about 20%, or about 25%, or about 35%, or about 50%, or about 80%, or about 90% or more (weight/weight). In various embodiments, the at least one ECM component comprises a quantity of AGE less than at least about 0.1%, at least about 0.5%, or at least about 1%, and may be as great as or more than about 5%, or about 10%, or about 15%, or about 20%, or about 25%, or about 35%, or about 50%, or about 80%, or about 90%. In various embodiments, the AGE is FFI, pentosidine, NFC-1, or malondialdehyde. In another embodiment, the ECM component is not substantially cross-linked.

In certain embodiments, the composition is a substantially pure solid or liquid. In other embodiments, the substantially pure solid or liquid comprises one or more ECM components selected from the group consisting of collagen, elastin, hyaluronic acid, chondroitin sulfate, heparan sulfate, laminin or fibronectin, and combinations thereof. In other embodiments, the composition is a substantially pure solid or liquid, substantially free of non-ECM components. In one embodiment, the composition is a substantially pure solid or liquid, substantially free of nucleic acid. In another embodiment, the substantially pure solid or liquid is substantially free of AGE. In other embodiments, the composition is a crude preparation solid or liquid. In one embodiment, the crude preparation is a whole cell extract. In other embodiments, the crude preparation comprises one or more ECM components selected from the group consisting of collagen, elastin, hyaluronic acid, chondroitin sulfate, heparan sulfate, laminin or fibronectin, and combinations thereof. In another embodiment, the crude preparation is substantially free of AGE.

In various embodiments, the composition comprises a solid powder, aqueous solution, partially emulsified aqueous solution, or emulsifications. In one embodiment, the aqueous solution is acidic. In one embodiment, the aqueous solution includes glycerin and ethanol. In another embodiment, partially emulsified aqueous solution, or emulsifications contain a proportion of aqueous solution and an oil component. In various embodiments, the oil component in compositions comprises at least about 0.3% or less to about 30% or more, such as at least about 0.5% to about 20% (weight/weight). In other embodiments, the oil components in compositions comprises at least about 0.5% to about 50%, such as 5% to 30% (weight/weight).

In various embodiments, the composition is mixed with additives in aqueous or oil components, comprising preservatives, pH adjusters, moisturizer, germicide, anti-inflammatory agent, dye, aromas, fragrances, antioxidants, ultraviolent absorbent, vitamin, alcohol, carbohydrates, or other components routinely used in skin care applications. In one embodiment, the preservative is sodium benzoate. In various embodiments, one or more additives is provided in compositions comprising at least about 0.0001%, at least about 0.01%, at least about 0.1%, at least about 0.5%, or at least about 1%, and may be as great as or more than about 5%, or about 10%, or about 15%, or about 20%, or about 25% or more (weight/weight).

In other embodiments, compositions are formulated with one or more ECM components as an active ingredient. In various embodiments, one or more ECM component provided in a composition comprises at least about 0.0001%, at least about 0.01%, at least about 0.1%, at least about 0.5%, or at least about 1%, and may be as great as or more than about 5%, or about 10%, or about 15%, or about 20%, or about 25% or more (weight/weight). In other embodiments, the cosmetically acceptable carrier in a composition comprises about 1% or less to about 99.9% or more, such as from about 10% to 90%, including about 25% to about 80% (weight/weight).

In various embodiments, the composition is formulated for topical application to the skin, such as the skin surrounding or comprising the eyes, mouth, nose, forehead, ears, neck, hands, feet, hair, and/or overall body. For example, the topical skin care composition may be in the form of a solution, serum, cream, lotion, body milk, emulsion, balm, gel, soap, conditioner, powder and the like. Alternatively, the topical skin care composition may be in the form of a shampoo, conditioner, serum, or toner. In other embodiments, the composition is formulated for topical application to hair or scalp.

In other embodiments, the composition is provided as an active ingredient in a composition formulated for topical application to the skin. In other embodiments, the composition is as provided as an active ingredient in a composition formulated for topical application to hair or scalp. In other embodiments, the composition is provided as an active ingredient in a composition formulated for cosmetic use. In other embodiments, the composition is provided as an active ingredient in a composition formulated for use as a treatment for a subject in need of treatment. Various skin-related conditions include appearance of aging, wrinkles, fine lines, thinness, diminished elasticity or suppleness, dry skin, undesirable apperance of pores, pronounced appearance of stretch marks and scars, undesiable color tone and hue, dermatitis, eczema, sunburn, inflammation, pruritic lesions, inflammatory and non-inflammatory lesions of the skin of a subject. Other conditions related to hair include baldness (i.e., alopecia), reduced shaft volume, structural deformations (e.g., split ends), low elasticity, brittleness, dullness, dryness, slow growth, among others.

The present invention further provide a method of preparing an exact from stem cells of a plant, fruit or vegetable source. In one embodiment, the method comprises isolating adult somatic cells from a plant, fruit or vegetable source, culturing the adult somatic cells on a solid medium containing components promoting dedifferentiation, inducing dedifferentiation of the adult somatic cells into a callus, disaggregating the callus into single cells in a liquid suspension medium. In another embodiment, the method further comprises homogenizing the liquid suspension into a broth extract and adding a liposome preparation.

In another embodiment, the method further comprises purification of at least one component from the extract.

### EXAMPLES

### Example 1

### Production of Stem Cell Clusters from Embryonic Stem Cells

This example describes the production of a population of SCCs cells from an established ES cell population. Similar methods can be used for producing SCCs from other murine ES cell lines.

The 129-SvEv-tac ES cell line #501 derived from 12956/SvEv-Taconic mice (Primogenix), is maintained in Dulbecco's modified Eagles medium (DMEM) supplemented with 15% fetal calf serum (FCS), 1.5×10⁻⁴ monothioglycerol (MTG), and leukemia inhibitory factor (LIF). The ES cells are passaged every 2-3 days at a dilution of approximately 1:15. Two days before the initiation of the differentiation cultures, undifferentiated ES cells are passaged into Iscove's modified Dulbecco's medium (IMDM) supplemented with the above components. Optionally, 50 μg/mL ascorbic acid may be introduced to increase matrix thickness and improved ECM yield. To induce differentiation into an SCC, the ES cells are trypsinized, washed, and counted using techniques standard in the art. The freshly dissociated ES cells are then cultured in IMDM containing 15% platelet-derived fetal bovine serum (PDS; obtained from Antech, Tex.; also referred to herein as platelet-poor fetal bovine serum, PP-FBS), 4.5×10⁻⁴ M MTG, transferrin (300 μg), glutamine (2 mM). The ES cells are plated in a final volume of 10 ml at a concentration of about 3000 to about 4500 cells per ml of medium in 150 mm bacterial grade dishes. The ES cell population is then cultured in a humidified environment of 5% CO₂, at a temperature of 37° C. After 3 days, SCCs are transferred back onto adherent plates and incubated in complete media with daily changes for an additional 12 days. The SCCs can be viewed under a Leitz inverted light microscope and will generally consist of groups of tightly packed cells, in which individual cells are not easily detectable.

### Example 2

### Extraction of Complete Extracellular Matrix from SCCs

This example describes the extraction of ECM from SCCs grown in T-150 flasks. SCCs generated in Example 1 are subjected to the following protocol. The culture flasks containing the SCCs are taken out of the incubator and the culture medium is carefully aspirated. The flasks are gently rinsed twice with 8 ml of PBS by touching the pipette against the flask wall. A solution of pre warmed (37° C.) extraction buffer (PBS containing 0.5% Triton X-100, 20 mM NH₄OH) is gently added using 5 ml/flask. Cell lysis is monitored by inspection with an inverted microscope. Flasks are incubated at 37° C. until no more intact cells are visible. Remaining cellular debris is diluted by slowly adding 3 ml of PBS, taking care not to disturb the newly formed and freshly denuded matrix. Flasks are stored overnight at 4° C. The diluted debris is carefully aspirated the next day leaving a thin liquid layer to keep the matrix hydrated at all times.

The matrix layer is rinsed with 6 ml of PBS by gently adding and aspirating while keeping the matrix hydrated. The matrix is treated briefly with a solution of 5 ml of DNase I prepared in PBS supplemented with 1 mM CaCl₂ and 1 mM MgSO₄ and incubated for 30 min at 37° C. The enzyme solution is aspirated and the matrix carefully rinsed with two washes with 8 ml of PBS, aspirating the excess of PBS after slightly tilting the flasks and carefully aspirating the PBS collected on one side of the flask. The flasks are put on ice and 5 ml of solubilization buffer is added (5 M guanidine-HCl containing 10 mM DTT). The matrix is harvested by scraping the flasks with a cell scraper to one side and pipetting the mixture into a plastic centrifuge tube. The flasks are rinsed with 3 ml of solubilization buffer, combining with the previously harvested matrix into the same tube. The matrix mixture is centrifuged at 12,000×g at 4° C. and the supernatant is saved. The supernatant is then dialyzed against 0.5 M acetic acid with four changes in one day. The final dialyzate is evaporated by lyophilization and resuspended in 1/10^{th} the original volume with 0.5 M acetic acid. A small sample ( 1/10^{th} volume) is taken and submitted for total protein mass, standard amino acid analysis and hydroxyproline and hydroxylysine content. The rest is stored at −20° C. until further use and formulation.

### Example 3

### Extraction of Collagen-Enriched Extracellular Matrix from SCCs

This example describes the extraction of a collagen-enriched fraction associated with the ECM from SCCs grown in T-150 flasks. SCCs generated in Example 1 are subjected to the following protocol. The culture flasks containing the SCCs are taken out of the incubator and the culture medium is carefully aspirated. The flasks are gently rinsed twice with 8 ml of PBS by touching the pipette against the flask wall. An ice-cold solution of 0.5 M acetic or lactic acid containing 0.1 mg/ml pepsin is gently added, using 5 ml/flask. Flasks are incubated at 4° C. for 24 hr on a rocking platform with gentle rotation. The extract is carefully harvested and transferred to a centrifuge plastic tube. Flasks are rinsed with 3 ml 0.5 M ice-cold acetic or lactic acid, collecting the remaining cells and insoluble materials with a cell scraper to one side of the flask. This mixture is combined with the harvested extract and then centrifuged at 12,000×g for 15 min at 4 C. The total collagen fraction may be concentrated using a salting out procedure by slowly adding NaCl to a final concentration of 0.9 M.

The mixture is incubated overnight at 4° C. and the resulting precipitate is collected by centrifugation at 12,000×g for 15 min at 4° C. The precipitate is dissolved in ice-cold 0.5 M acetic or lactic acid and dialyzed against the same with four changes in one day. The final dialyzate may be evaporated by lyophilization and resuspended in 1/10^{th} the original volume with 0.5 M lactic or acetic acid. A small sample ( 1/10^{th} volume) is taken and submitted for total protein mass, standard amino acid analysis and hydroxyproline and hydroxylysine content. The rest is stored at −20° C. until further use and formulation.

### Example 4

### Extracts Prepared from Stem Cells from Plant, Vegetable and Fruit Sources

This example describes preparation of extracts from stem cells obtained from a plant, vegetable or fruit source. Adult somatic cells may be isolated from a plant, vegetable, or fruit organism and placed on solid medium in a culture vessel, wherein the solid medium contains components promoting the dedifferentiation of the adult somatic cells. Following induction of the dedifferentiation process through formation of a callus, the callus may be mechanically or chemically dissociated as single cells to be grown in suspension in liquid medium. Suspension cultures may require additional cultivation steps for scale up purposes. Extracts may be prepared from stem cell cultures through combining a homogenized whole cell broth with a liposome preparation for solubilization. Addition of a liposome component to the extract further provides an oil component to the aqueous solution, wherein various agents and carriers related to the use of cosmetics may be added (e.g., preservatives, stabilizers, antioxidants).

### Example 5

### Use of a Composition Containing Stem Cell Extracts for Anti-Wrinkle Treatment

This example describes the use of a composition containing stem cell extracts for anti-aging and anti-wrinkle treatment of the skin. The composition can appear as a cream, lotion, gel, toner, serum, or in other forms ordinarily known to be utilized for application of anti-wrinkle treatments. A quantity of the composition, for example 1 ml to 100 mL or more, is applied topically to a site of interest, such as the face or hands. Application may occur through obtaining a quantity of composition from a suitable container using a finger, squeezing the composition onto the skin surface, or directly applied through an applicator such as a pump. As necessary, the composition is spread over and/or rubbed into the site using hands or fingers, or a suitable device, such as an applicator tip. The composition may contain various components suitable for enhancing application to the skin area, such as ethanol to promote drying through evaporation or glycerin to promote spreading on the skin surface. The composition may be applied singularly or repeatedly as is necessary to achieve effect of anti-wrinkling effects, such as a reduction in appearance of fine lines and wrinkles, or anti-aging effects, such as improved tonicity and color on the skin.

### Example 6

### Use of a Composition Containing Stem Cell Extracts for Improving Appearance of Hair

This example describes the use of a composition containing stem cell extracts to improve the appearance of hair. The composition can be in the form of a shampoo, conditioner, serum, or in other forms ordinarily known to be utilized for application of hair treatments. A quantity of the composition, for example 1 ml to 100 mL or more, is applied topically to a site of interest, such as onto the hair surface or directly onto the scalp. Application may occur in association with a shower or bath, wherein the composition is massaged and rubbed into the hair and/or scalp and rinsed out using water, or may be a “leave-in” treatment, wherein the composition is applied to wet or dry hair and left in-place for an extended period before being removed by rinsing. The composition may be applied singularly or repeatedly as is necessary to achieve the effect of improved hair appearance, as demonstrated by increased size/volume of individual hairs, improved hair structure (e.g., fewer split ends at hair termini), or better elastic and mechanical properties. In related applications, the composition can be applied to the skin of the scalp for the purpose of reducing or eliminating hair loss, by promoting maintenance and regenerative mechanisms of skin cells which are involved with the routine growth and replacement of hair.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the sources of ECM and constituent products, the manufacturing techniques used to create cosmetic products, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method for obtaining at least one extracellular matrix component, the method comprising the steps of:
culturing mammalian embryonic stem cells (“ESCs”) to form a culture of ESCs; and
extracting from the culture of ESCs or differentiated ESCs the at least one extracellular matrix component.

2. The method of claim 1, wherein the mammalian ESCs are murine ESCs.

3. The method of claim 1, wherein the mammalian ESCs are cultured on feeder cells.

4. The method of claim 1, further comprising the step of inducing differentiation in the culture of ESCs prior to the step of extraction.

5. The method of claim 4, further comprising treatment of the culture of ESCs with dispase or collagenase prior to the step of inducing.

6. The method of claim 4, wherein the inducing step further comprises transferring the culture of ESCs to a container under conditions which reduce the likelihood of adherence of the culture of ESCs to a surface of the container.

7. The method of claim 4, wherein the inducing step further comprises inducing the culture of ESCs in a media solution.

8. The method of claim 6, further comprising the step of rocking the container containing the culture of ESCs.

9. The method of claim 4, wherein the inducing step further comprises inducing the culture of ESCs in a hanging drop.

10. The method of claim 4, wherein the step of extracting further comprises treating the culture of ESCs with a salt, a detergent and/or an acid, and separating the culture of ESCs from the at least one extracellular matrix component.

11. The method of claim 10, further comprising the step of purifying the at least one extracted extracellular matrix component.

12. The method of claim 11, wherein the step of purifying further comprises centrifugation, chromatography, precipitation, filtration and/or organic solvent extraction.

13. The method of claim 11, further comprising the step of lyophilizing the at least one extracted extracellular matrix component.

14. The method of claim 1, wherein the at least one extracellular matrix component is a collagen.

15. The method of claim 14, further comprising treatment of the at least one extracellular matrix component with a protease.

16. The method of claim 1, wherein the at least one extracellular matrix component is a proteoglycan.

17. The method of claim 1, wherein the at least one extracellular matrix component is elastin.

18. The method of claim 1, wherein the at least one extracellular matrix component is laminin or fibronectin.

19. A composition comprising:
at least one extracellular matrix component extracted from a culture of mammalian embryonic stem cells (“ESCs”) or differentiated ESCs; and
a cosmetically-acceptable carrier.

20. The composition of claim 19, wherein the mammalian ESCs are murine ESCs.

21. The composition of claim 19, wherein the mammalian ESCs are cultured on feeder cells.

22. The composition of claim 19, wherein the at least one extracellular matrix component is produced by a process, comprising:
culturing ESCs to form a culture of ESCs;
inducing the ESCs to differentiate; and
extracting from the culture of differentiated ESCs the at least one extracellular matrix component.

23. The composition of claim 22, further comprising treatment of the culture of ESCs with dispase or collagenase prior to the step of inducing.

24. The composition of claim 22, wherein the inducing step further comprises transferring the culture of ESCs to a container under conditions which reduce the likelihood of adherence of the culture of ESCs to a surface of the container.

25. The composition of claim 22, wherein the inducing step further comprises inducing the culture of ESCs in a media solution.

26. The composition of claim 22, wherein the step of extracting further comprises treating the culture of ESCs with a salt, a detergent and/or an acid, and separating the culture of ESCs from the at least one extracellular matrix component.

27. The composition of claim 26, further comprising the step of purifying the at least one extracted extracellular matrix component.

28. The composition of claim 27, wherein the step of purifying further comprises centrifugation, chromatography, precipitation, filtration and/or organic solvent extraction.

29. The composition of claim 27, further comprising the step of lyophilizing the at least one extracted extracellular matrix component.

30. The composition of claim 19, wherein the at least one extracellular matrix component is a collagen.

31. The composition of claim 30, further comprising treatment of the at least one extracellular matrix component with a protease.

32. The composition of claim 19, wherein the at least one extracellular matrix component is a proteoglycan.

33. The composition of claim 19, wherein the at least one extracellular matrix component is elastin.

34. The composition of claim 19, wherein the at least one extracellular matrix component is laminin or fibronectin.

35. A method of manufacturing a composition comprising the steps of:
providing at least one extracellular matrix component extracted from a culture of mammalian embryonic stem cells (“ESCs”) or differentiated ESCs; and
adding a cosmetically-acceptable carrier to the at least one extracellular matrix component.

36. The method of claim 35, wherein the mammalian ESCs are murine ESCs.

37. The method of claim 35, wherein the at least one extracellular matrix component is a collagen.

38. The method of claim 35, wherein the at least one extracellular matrix component is a proteoglycan.

39. The method of claim 35, wherein the at least one extracellular matrix component is elastin.

40. The method of claim 35, wherein the at least one extracellular matrix component is laminin or fibronectin.

41. (canceled)

42. (canceled)

43. (canceled)

44. (canceled)

45. (canceled)

46. (canceled)

47. (canceled)

48. (canceled)
